(19) United States
(12) Patent Application Publication
(10) Pub. No.: US 2010/0262013 A1
(43) Pub. Date: Oct. 14, 2010

(54) Universal Multiple Aperture Medical Ultrasound Probe

(76) Inventors: David M. Smith, Lodi, CA (US); Sharon L. Adam, San Jose, CA (US); Donald F. Specht, Los Altos, CA (US); John P. Lunsford, Los Altos Hills, CA (US); Kenneth D. Brewer, Santa Clara, CA (US)

Correspondence Address: SHAY GLENN LLP, 2755 CAMPUS DRIVE, SUITE 210, SAN MATEO, CA, 94403, US

(21) Appl. No.: 12/760,375

(22) Filed: Apr. 14, 2010

Related U.S. Application Data
(60) Provisional application No. 61/169,251, filed on Apr. 14, 2009.
(60) Provisional application No. 61/169,221, filed on Apr. 14, 2009.

(51) Int. Cl. A61B 8/14

(52) U.S. Cl. 600/459

(57) ABSTRACT

A Multiple Aperture Ultrasound Imaging (MAUI) probe or transducer is uniquely capable of simultaneous imaging of a region of interest from separate physical apertures. Construction of probes can vary by medical application. That is, a general radiology probe can contain multiple transducers that maintain separate physical points of contact with the patient's skin, allowing multiple physical apertures. A cardiac probe may contain only two transmitters and receivers where the probe fits simultaneously between two or more intracostal spaces. An intracavity version of the probe can space transmit and receive transducers along the length of the wand, while an intravenous version can allow transducers to be located on the distal length the catheter and separated by mere millimeters. Algorithms can solve for variations in tissue speed of sound, thus allowing the probe apparatus to be used virtually anywhere in or on the body.

### CROSS REFERENCE TO RELATED APPLICATIONS

This application claims the benefit under 35 U.S.C. 119 of U.S. Provisional Patent Application No. 61/169,251, filed Apr. 14, 2009, titled “Universal Multiple Aperture Medical Ultrasound Transducer”, and U.S. Provisional Patent Application No. 61/169,221, filed Apr. 14, 2009, titled “Multi Aperture Cable Assembly for Multiple Aperture Probe for Use in Medical Ultrasound.”

This application is related to U.S. patent application Ser. No. 11/865,501, filed Oct. 1, 2007, titled “Method and Apparatus to Produce Ultrasonic Images Using Multiple Apertures”, U.S. patent application Ser. No. 11/532,013, filed Sep. 14, 2006, titled “Method and Apparatus to Visualize the Coronary Arteries Using Ultrasound”, U.S. Provisional Patent Application No. 61/305,784, filed Feb. 18, 2010, titled “Alternative Method for Medical Multi-Aperture Ultrasound Imaging”, and PCT Application No. PCT/US2009/053096, filed Aug. 7, 2009, titled “Imaging with Multiple Aperture Medical Ultrasound and Synchronization of Add-on Systems”. These applications are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

The present invention relates generally to imaging techniques used in medicine, and more particularly to medical ultrasound, and still more particularly to an apparatus for producing ultrasonic images using multiple apertures.

### BACKGROUND OF THE INVENTION

In conventional ultrasonic imaging, a focused beam of ultrasound energy is transmitted into body tissues to be examined and the returned echoes are detected and plotted to form an image. In echocardiography, the beam is usually stepped in increments of angle from a center probe position, and the echoes are plotted along lines representing the paths of the transmitted beams. In abdominal ultrasonography, the beam is usually stepped laterally, generating parallel beam paths, and the returned echoes are plotted along parallel lines representing these paths. The following description will relate to the angular scanning technique for echocardiography and general radiology (commonly referred to as a sector scan). However, the same concept with minor modifications can be implemented in any ultrasound scanner.

The basic principles of conventional ultrasonic imaging are described in the first chapter of Echocardiography, by Harvey Feigenbaum (Lippincott Williams & Wilkins, 5th ed., Philadelphia, 1993). It is well known that the average velocity υ of ultrasound in human tissue is about 1540 m/sec, the range in soft tissue being 1440 to 1670 m/sec (P. N. T. Wells, Biomedical Ultrasonics, Academic Press, London, New York, San Francisco, 1977). Therefore, the depth of an impedance discontinuity generating an echo can be estimated as the round-trip time for the echo multiplied by v/2, and the amplitude is plotted at that depth along a line representing the path of the beam. After this has been done for all echoes along all beam paths, an image is formed. The gaps between the scan lines are typically filled in by interpolation.

In order to insonify the body tissues, a beam formed either by a phased array or a shaped transducer is scanned over the tissues to be examined. Traditionally, the same transducer or array is used to detect the returning echoes. This design configuration lies at the heart of one of the most significant limitations in the use of ultrasonic imaging for medical purposes; namely, poor lateral resolution. Theoretically the lateral resolution could be improved by increasing the aperture of the ultrasonic probe, but the practical problems involved with aperture size increase have kept apertures small and lateral resolution large. Unquestionably, ultrasonic imaging has been very useful even with this limitation, but it could be more effective with better resolution.

In the practice of cardiology, for example, the limitation on single aperture size is dictated by the space between the ribs (the intercostal spaces). For scanners intended for abdominal and other use (e.g. intracavity or intravenous), the limitation on aperture size is a serious limitation as well. The problem is that it is difficult to keep the elements of a large aperture array in phase because the speed of ultrasound transmission varies with the type of tissue between the probe and the area of interest. According to Wells (Biomedical Ultrasonics, as cited above), the transmission speed varies up to plus or minus 10% within the soft tissues. When the aperture is kept small, the intervening tissue is, to a first order of approximation, all the same and any variation is ignored. When the size of the aperture is increased to improve the lateral resolution, the additional elements of a phased array may be out of phase and may actually degrade the image rather than improving it.

In the case of cardiology, it has long been thought that extending the phased array into a second or third intercostal space would improve the lateral resolution, but this idea has met with two problems. First, elements over the ribs have to be eliminated, leaving a sparsely filled array and new theory would be required to steer the beam emanating from such an array. Second, the tissue speed variation described above, would need to be compensated.

In the case of abdominal imaging, it has also been recognized that increasing the aperture size could improve the lateral resolution. Although avoiding the ribs is not a problem, beam forming using a sparsely filled array and, particularly, tissue speed variation needs to be compensated. With single aperture transducers, it has been commonly assumed that the beam paths used by the elements of the transducer are close enough together to be considered similar in tissue density profile, and therefore that no compensation was necessary. The use of this assumption, however, severely limits the size of the aperture that can be used. The method of compensation taught in U.S. patent application Ser. No. 11/865,501, filed on Oct. 1, 2007, titled “Method and Apparatus to Produce Ultrasonic Images Using Multiple Apertures” may be advantageously applied in groups of or individually to the receive elements in order to make effective use of wide or multiple aperture configurations. Further solutions, described herein, are desirable in order to overcome the various shortcomings in the conventional art as outlined above in order to maintain information from an extended phased array “in phase”, and to achieve a desired level of imaging lateral resolution.

### SUMMARY OF THE INVENTION

A multi-aperture ultrasound probe is provided, comprising a probe shell, a first ultrasound transducer array disposed in the shell and having a plurality of transducer elements, wherein at least one of the plurality of transducer elements of the first ultrasound transducer array is configured to transmit an ultrasonic pulse, a second ultrasound transducer array disposed in the shell and being physically separated from the first ultrasound transducer array, the second ultrasound transducer array having a plurality of transducer elements, wherein at least one of the plurality of transducer elements of the second ultrasound transducer array is configured to receive an echo return of the ultrasonic pulse.

In some embodiments, the second ultrasound transducer array is angled towards the first ultrasound transducer array. In other embodiments, the second ultrasound transducer array is angled in the same direction as the first ultrasound transducer array.

In some embodiments, at least one of the plurality of transducer elements of the first ultrasound transducer array is configured to receive an echo return of the ultrasonic pulse. In other embodiments, at least one of the plurality of transducer elements of the second ultrasound transducer array is configured to transmit an ultrasonic pulse. In additional embodiments, at least one of the plurality of transducer elements of the second ultrasound transducer array is configured to transmit an ultrasonic pulse.

In some embodiments, the shell further comprises an adjustment mechanism configured to adjust the distance between the first and second ultrasound transducer arrays.

In another embodiment, the probe comprises a third ultrasound transducer array disposed in the shell and being physically separated from the first and second ultrasound transducer arrays, the third ultrasound transducer array having a plurality of transducer elements, wherein at least one of the plurality of transducer elements of the third ultrasound transducer array is configured to receive an echo return of the ultrasonic pulse.

In some embodiments, the first ultrasound transducer array is positioned near the center of the shell and the second and third ultrasound transducer arrays are positioned on each side of the first ultrasound transducer array. In other embodiments, the second and third ultrasound transducer arrays are angled towards the first ultrasound transducer array.

In some embodiments, the first ultrasound transducer array is recessed within the shell. In another embodiment, the first ultrasound transducer array is recessed within the shell to be approximately aligned with an inboard edge of the second and third ultrasound transducer arrays.

In other embodiments, the first, second, and third ultrasound transducer arrays each comprise a lens that forms a seal with the shell. In some embodiments, the lenses form a concave arc.

In another embodiment, a single lens forms an opening for the first, second, and third ultrasound transducer arrays.

The probe can be sized and configured to be inserted into a number of different patient cavities. In some embodiments, the shell is sized and configured to be inserted into an esophagus of a patient. In another embodiment, the shell is sized and configured to be inserted into a rectum of a patient. In another embodiment, the shell is sized and configured to be inserted into a vagina of a patient. In yet another embodiment, the shell is sized and configured to be inserted into a vessel of a patient.

In some embodiments, the plurality of transducer elements of the first ultrasound transducer can be grouped and phased to transmit a focused beam. In another embodiment, at least one of the plurality of transducer elements of the first ultrasound transducer are configured to produce a semicircular pulse to insonify an entire slice of a medium. In yet another embodiment, at least one of the plurality of transducer elements of the first ultrasound transducer are configured to produce a semispherical pulse to insonify an entire volume of the medium.

In some embodiments, the first and second transducer arrays include separate backing blocks. In other embodiments, the first and second transducer arrays further comprise a flex connector attached to the separate backing blocks.

Some embodiments of the multi-aperture ultrasound probe further comprise a probe position displacement sensor configured to report a rate of angular rotation and lateral movement to a controller.

In other embodiments, the first ultrasound transducer array comprises a host ultrasound probe, and the multi-aperture ultrasound probe further comprises a transmit synchronizer device configured to report a start of transmit from the host ultrasound probe to a controller.

### BRIEF DESCRIPTION OF THE DRAWINGS

FIG. 1 illustrates a two-aperture system.

FIG. 2 illustrates a three-aperture system.

FIG. 3 is a schematic diagram showing a possible fixture for positioning an omni-directional probe relative to the main (insonifying) probe.

FIG. 4 is a schematic diagram showing a non-instrumented linkage for two probes.

FIG. 5 is a block diagram of the transmit and receive functions where a Multiple Aperture Ultrasound Transducer is used in conjunction with an add-on instrument. In this embodiment, the center probe is used for transmit only and mimics the normal operation of the host transmit probe.

FIG. 5a is a block diagram of the transmit and receive functions where a Multiple Aperture Ultrasound Transducer is used in a two transducer array format, primarily for cardiac applications, with an add-on instrument. In this case, one probe is used for transmit only and mimics the normal operation of the host transmit probe, while the other probe operates only as a receiver.

FIG. 6 is a block diagram of the transmit and receive functions where a Multiple Aperture Ultrasound Transducer is used in conjunction with only a Multiple Aperture Ultrasonic Imaging (MAUI) device. The stand-alone MAUI electronics control all elements on all apertures. Any element may be used as a transmitter or omni-receiver, or grouped into transmit and receive full apertures or even sub-arrays.

FIG. 6a is a block diagram demonstrating that the MAUI electronics can utilize elements on outer apertures of the probe to transmit not only to improve image quality, but also to see around objects in the near field such as a vertebral structure.

FIGS. 6b and 6c are block diagrams demonstrating the ability of MAUI electronics to alternate transmissions between apertures. This ability gets more energy to the targets closer to each aperture while still enjoying the full benefit of the wide aperture.

FIG. 7a is a schematic perspective view showing an adjustable, extendable hand held two-aperture probe (especially adapted for use in cardiology US imaging). This view shows the probe in a partially extended configuration.

FIG. 7b is a side view in elevation thereof showing the probe in a collapsed configuration.

FIG. 7c shows the probe extended so as to place the heads at a maximum separation distance permitted under the probe design, and poised for pushing the separated probe apertures into a collapsed configuration.

FIG. 7d is a side view in elevation again showing the probe in a collapsed configuration, with adjustment means shown (i.e., as scroll wheel).

FIG. 7e is a detailed perspective view showing the surface features at the gripping portion of the probe.

FIG. 8 illustrates a hand-held two aperture probe that is constructed with arrays configured in a horizontal plane, at a fixed width and is not adjustable.

FIG. 8a illustrates a hand-held two aperture probe that is constructed with two arrays canted inward at an angle. The probe illustrated has a fixed width and is not adjustable.

FIG. 9 illustrates individual elements in each of the apertures in a multi-aperture probe containing three or more arrays. The illustration shows elements of a sub-array being used for transmission while all elements on every aperture are used to receive.

FIG. 9a illustrates elements of a sub-array being used for transmit from the furthest most aperture, while all elements on every other aperture receive. Elements can operate singularly, in sub-arrays or as an entire array while transmitting or receiving.

FIG. 9b illustrates individual elements in each of the apertures in a multi-aperture probe containing only two arrays. The illustration shows elements of a sub-array being used for transmission while all elements on both aperture are used to receive.

FIG. 9c illustrates alternate elements of a sub-array being used during transmission while all elements on both apertures are used to receive.

FIG. 10 is a diagram showing a multi-aperture probe with center array recessed from the skin line to a point in line with the trailing edges the outboard arrays, a concaved unified lens and the outboard arrays canted at an angle. FIG. 10 includes a transmit synchronizer module and probe position displacement sensor.

FIG. 10a is a diagram showing the multi-aperture probe lenses view with the center array recessed to a point in line with the trailing edges the outboard arrays, the two outboard arrays canted at an angle.

FIG. 11 is a diagram of a multi-aperture probe configuration with arrays configured in a horizontal plane. FIG. 11 includes a transmit synchronizer module and probe position displacement sensor.

FIG. 11a is a diagram showing the lenses of the multi-aperture probe with its center array and outboard arrays mounted in the same plane.

FIG. 12 is a diagram showing a multi-aperture probe with center array recessed from the skin line to a point in line with the trailing edges the outboard arrays, a unified lens and the outboard arrays canted at an angle. FIG. 12 includes a transmit synchronizer module and probe position displacement sensor.

FIG. 12a is a diagram showing the multi-aperture probe lens view with the center array recessed from the skin line to a point in line with the trailing edges the outboard arrays, the two outboard arrays canted at an angle and a unified lens.

FIG. 13 illustrates of a multi-aperture omniplane style transesophogeal (TEE) probe using three or more arrays. The top view is of the apertures as seen through the lens at the distal end of the probe. The arrays illustrated here are using a common backing plate, even though each would utilize its own backing block and lens.

FIG. 13a illustrates of a multi-aperture omniplane style transesophogeal (TEE) probe using only two arrays. The top view is of the apertures as seen through the lens at the distal end of the probe. The arrays illustrated here are using a common backing plate, even though each would utilize its own backing block and lens.

FIG. 14 illustrates a multi-aperture endo rectal probe using three apertures where the center array is recessed from to a point in line with the trailing edges the outboard arrays, a unified lens is provided on the external encasement, and the outboard arrays canted at an angle.

FIG. 14a illustrates a multi-aperture endo rectal probe using only two aperture. A unified lens is provided on the external encasement, and the arrays are canted at an angle.

FIG. 15 illustrates a multi-aperture endo vaginal probe using three apertures where the center array is recessed from to a point in line with the trailing edges the outboard arrays, a unified lens is provided on the external encasement, and the outboard arrays canted at an angle.

FIG. 15a illustrates a multi-aperture endo vaginal probe using only two aperture. A unified lens is provided on the external encasement, and the arrays are canted at an angle.

FIG. 16 illustrates a multi-aperture intravenous ultrasound probe (IVUS) using three apertures where the center array is recessed from to a point in line with the trailing edges the outboard arrays, a unified lens is provided on the external encasement, and the outboard arrays canted at an angle.

FIG. 16a illustrates a multi-aperture intravenous ultrasound probe (IVUS) using only two aperture. A unified lens is provided on the external encasement, and the arrays are canted at an angle.

FIG. 17 illustrates three one-dimensional (1D) arrays for use in a multiple aperture ultrasound probe where the ultrasound crystal elements are formed by cutting or shaping the crystals linearly. Each crystal is placed on its own backing block, as is demonstrated here, physically separate from the other transducers prior to being placed in a probe encasement or onto a shared backing plate.

FIG. 17a illustrates two one-dimensional (1D) arrays for use in a multiple aperture ultrasound probe where the ultrasound crystal elements are formed by cutting or shaping the crystals linearly. Each crystal is place on its own backing block, as is demonstrated here, physically separate from the other transducers prior to being placed in a probe encasement or onto a shared backing plate.

FIG. 17b illustrates three one and half dimensional (1.5D) arrays for use in a multiple aperture ultrasound probe where the ultrasound crystal elements are formed by cutting or shaping the crystals transversely and then longitudinally so as to create rows. The longitudinal cuts are essential in creating improved transverse focus. Each crystal is placed on its own backing block, as is demonstrated here, physically separate from the other transducers prior to being placed in a probe encasement or onto a shared backing plate.

FIG. 17c illustrates two one and half dimensional (1.5D) arrays for use in a multiple aperture ultrasound probe where the ultrasound crystal elements are formed by cutting or shaping the crystals transversely and then longitudinally so as to create rows. The longitudinal cuts are essential in creating improved transverse focus. Each crystal is placed on its own backing block, as is demonstrated here, physically separate from the other transducers prior to being placed in a probe encasement or onto a shared backing plate.

FIG. 17d illustrates three matrix (2D) arrays were the crystals elements are formed by cutting or shaping the crystals into individual elements that can be individually activated or activated in groups. The cut or shaping of the elements is not specific to a single scan plan or dimension. Each crystal is placed on its own backing block, as is demonstrated here, physically separate from the other transducers prior to being placed in a probe encasement or onto a shared backing plate.

FIG. 17e illustrates two matrix (2D) arrays were the crystals elements are formed by cutting or shaping the crystals into individual elements that can be individually activated or activated in groups. The cut or shaping of the elements is not specific to a single scan plan or dimension. Each crystal is placed on its own backing block, as is demonstrated here, physically separate from the other transducers prior to being placed in a probe encasement or onto a shared backing plate.

FIG. 17f illustrates three arrays manufactured using Capacitive Micromachined Ultrasonic Transducers (CMUT). Each CMUT element can be individually activated or activated in groups. The size and shape of the total transducer array is unlimited even though elements usually share the same lens. Here, three rectangular arrays have been assembled on separate backing blocks, physically separated from other CMUT arrays prior to being place in a Multiple Aperture Transducer shell or shared backing plate.

FIG. 17g illustrates two arrays manufactured using Capacitive Micromachined Ultrasonic Transducers (CMUT). Each CMUT element can be individually activated or activated in groups. The size and shape of the total transducer array is unlimited even though elements usually share the same lens. Here, three rectangular arrays have been assembled on separate backing blocks, physically separated from other CMUT arrays prior to being place in a Multiple Aperture Transducer shell or shared backing plate.

FIG. 18 illustrates five arrays for use in a multiple aperture ultrasound probe where. Each crystal is placed on its own backing block, as is demonstrated here, physically separate from the other transducers prior to being placed in a probe encasement or onto a shared backing plate.

### DETAILED DESCRIPTION OF THE INVENTION

A Multiple Aperture Ultrasound Imaging (MAUI) Probe or Transducer can vary by medical application. That is, a general radiology probe can contain multiple transducers that maintain separate physical points of contact with the patient's skin, allowing multiple physical apertures. A cardiac probe may contain as few as two transmitters and receivers where the probe fits simultaneously between two or more intercostal spaces. An intracavity version of the probe, will space transmit and receive transducers along the length of the wand, while an intravenous version will allow transducers to be located on the distal length the catheter and separated by mere millimeters. In all cases, operation of multiple aperture ultrasound transducers can be greatly enhanced if they are constructed so that the elements of the arrays are aligned within a particular scan plane.

One aspect of the invention solves the problem of constructing a multiple aperture probe that functionally houses multiple transducers which may not be in alignment relative to each other. The solution involves bringing separated elements or arrays of elements into alignment within a known scan plane. The separation can be a physical separation or simply a separation in concept wherein some of the elements of the array can be shared for the two (transmitting or receiving) functions. A physical separation, whether incorporated in the construction of the probe's casing, or accommodated via an articulated linkage, is also important for wide apertures to accommodate the curvature of the body or to avoid non-echogenic tissue or structures (such as bone).

Any single omni-directional receive element (such as a single crystal pencil array) can gather information necessary to reproduce a two-dimensional section of the body. In some embodiments, a pulse of ultrasound energy is transmitted along a particular path; the signal received by the omni-directional probe can be recorded into a line of memory. When the process for recording is complete for all of the lines in a sector scan, the memory can be used to reconstruct the image.

In other embodiments, acoustic energy is intentionally transmitted to as wide a two-dimensional slice as possible. Therefore all of the beam formation must be achieved by the software or firmware associated with the receive arrays. There are several advantages to doing this: 1) It is impossible to focus tightly on transmit because the transmit pulse would have to be focused at a particular depth and would be somewhat out of focus at all other depths, and 2) An entire two-dimensional slice can be insonified with a single transmit pulse.

Omni-directional probes can be placed almost anywhere on or in the body: in multiple or intercostal spaces, the suprasternal notch, the substernal window, multiple apertures along the abdomen and other parts of the body, on an intracavity probe or on the end of a catheter.

The construction of the individual transducer elements used in the apparatus is not a limitation of use in multi-aperture systems. Any one, one and a half, or two dimensional crystal arrays (1D, 1.5D, 2D, such as a piezoelectric array) and all types of Capacitive Micromachined Ultrasonic Transducers (CMUT) can be utilized in multi-aperture configurations to improve overall resolution and field of view.

Transducers can be placed either on the image plane, off of it, or any combination. When placed away from the image plane, omni-probe information can be used to narrow the thickness of the sector scanned. Two dimensional scanned data can best improve image resolution and speckle noise reduction when it is collected from within the same scan plane.

Greatly improved lateral resolution in ultrasound imaging can be achieved by using probes from multiple apertures. The large effective aperture (the total aperture of the several sub apertures) can be made viable by compensation for the variation of speed of sound in the tissue. This can be accomplished in one of several ways to enable the increased aperture to be effective rather than destructive.

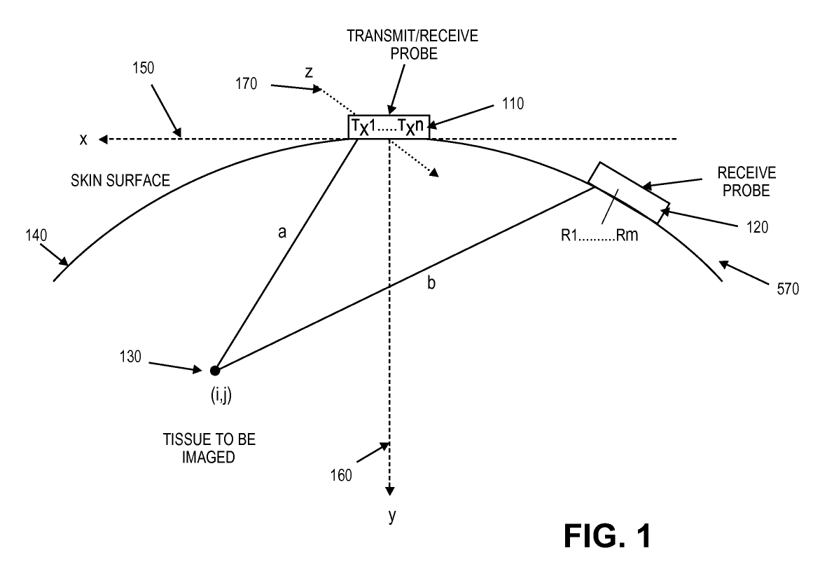

The simplest multi-aperture system consists of two apertures, as shown in FIG. 1. One aperture could be used entirely for transmit elements 110 and the other for receive elements 120. Transmit elements can be interspersed with receive elements, or some elements could be used both for transmit and receive. In this example, the probes have two different lines of sight to the tissue to be imaged 130. That is, they maintain two separate physical apertures on the surface of the skin 140. Multiple Aperture Ultrasonic Transducers are not limited to use from the surface of the skin, they can be used anywhere in or on the body to include intracavity and intravenous probes. In transmit/receive probe 110, the positions of the individual elements Tₓ1 through Tₓn can be measure in three different axes. This illustration shows the probe perpendicular to the x axis 150, so each element would have a different position x and the same position y on the y axis 160. However, the y axis positions of elements in probe 120 would be different since it is angled down. The z axis 170 comes in or out of the page and is very significant in determine whether an element is in or out of the scan plane.

Referring to FIG. 1, suppose that a Transmit Probe containing ultrasound transmitting elements T1, T2, . . . Tn 110 and a Receive Probe 120 containing ultrasound receive elements R1, R2, Rm are placed on the surface of a body to be examined (such as a human or animal). Both probes can be sensitive to the same plane of scan, and the mechanical position of each element of each probe is known precisely relative to a common reference such as one of the probes. In one embodiment, an ultrasound image can be produced by insonifying the entire region to be imaged (e.g., a plane through the heart, organ, tumor, or other portion of the body) with a transmitting element (e.g., transmit element Tₓ1), and then “walking” down the elements on the Transmit probe (e.g., Tₓ2, . . . Tₓn) and insonifying the region to be imaged with each of the transmit elements. Individually, the images taken from each transmit element may not be sufficient to provide a high resolution image, but the combination of all the images can provide a high resolution image of the region to be imaged. Then, for a scanning point represented by coordinates (i,j) it is a simple matter to calculate the total distance “a” from a particular transmit element Tₓn to an element of tissue at (i,j) 130 plus the distance “b” from that point to a particular receive element. With this information, one could begin rendering a map of scatter positions and amplitudes by tracing the echo amplitude to all of the points for the given locus.

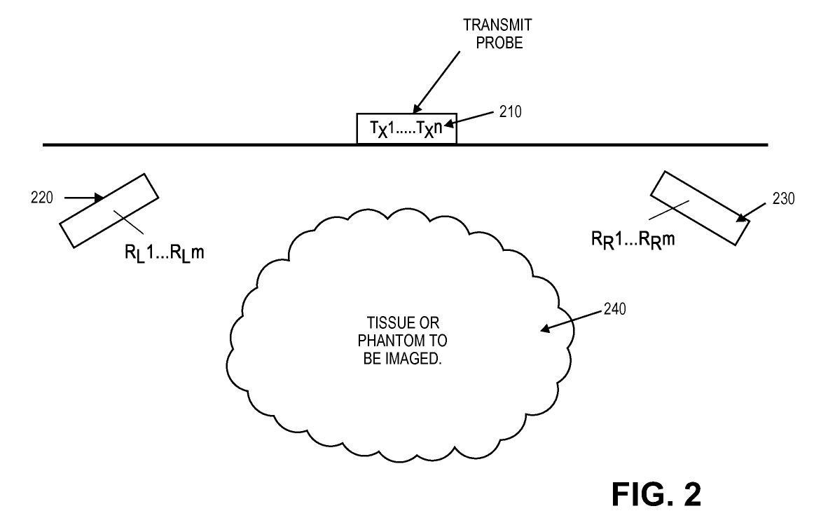

Another multi-aperture system is shown FIG. 2 and consists of transducer elements in three apertures. In one concept, elements in the center aperture 210 can be used for transmit and then elements in the left 220 and right 230 apertures can be used for receive. Another possibility is that elements in all three apertures can be used for both transmit and receive, although the compensation for speed of sound variation would be more complicated under these conditions. Positioning elements or arrays around the tissue to be imaged 240 provides much more data than simply having a single probe 210 over the top of the tissue.

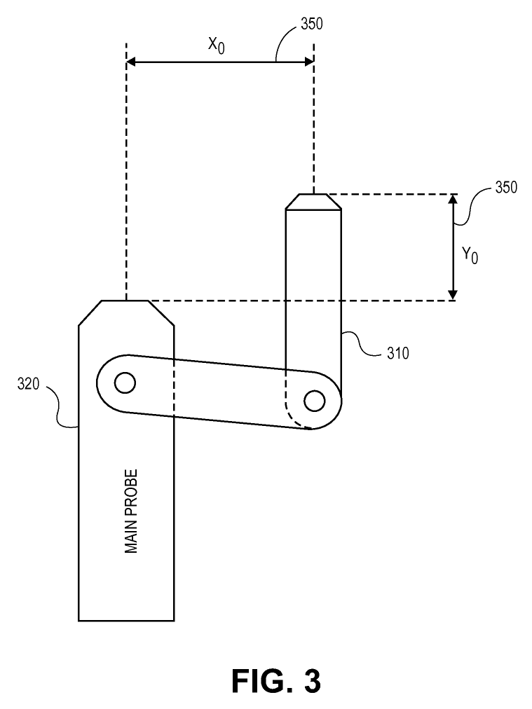

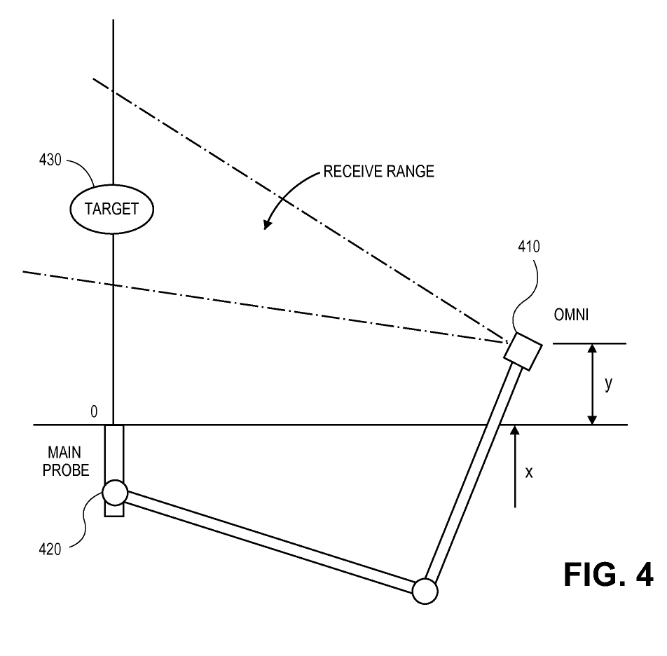

The Multiple Aperture Ultrasonic Imaging methods described herein are dependent on a probe apparatus that allows the position of every element to be known and reports those positions to any new apparatus the probe becomes attached. FIGS. 3 and 4 demonstrate how a single omni-probe 310 or 410 can be attached to a main transducer (phased array or otherwise) so as to collect data, or conversely, to act as a transmitter where the main probe then becomes a receiver. In both of these embodiments the omni-probe is already aligned within the scan plan. Therefore, only the x and y positions 350 need be calculated and transmitted to the processor. It is also possible to construct a probe with the omni-probe out of the scan plane for better transverse focus.

An aspect of the omni-probe apparatus includes returning echoes from a separate relatively non-directional receive transducer 310 and 410 located away from the insonifying probe transmit transducer 320 and 420, and the non-directional receive transducer can be placed in a different acoustic window from the insonifying probe. The omni-directional probe can be designed to be sensitive to a wide field of view for this purpose.

The echoes detected at the omni-probe may be digitized and stored separately. If the echoes detected at the omni-probe (310 in FIGS. 3 and 410 in FIG. 4) are stored separately for every pulse from the insonifying transducer, it is surprising to note that the entire two-dimensional image can be formed from the information received by the one omni. Additional copies of the image can be formed by additional omni-directional probes collecting data from the same set of insonifying pulses.

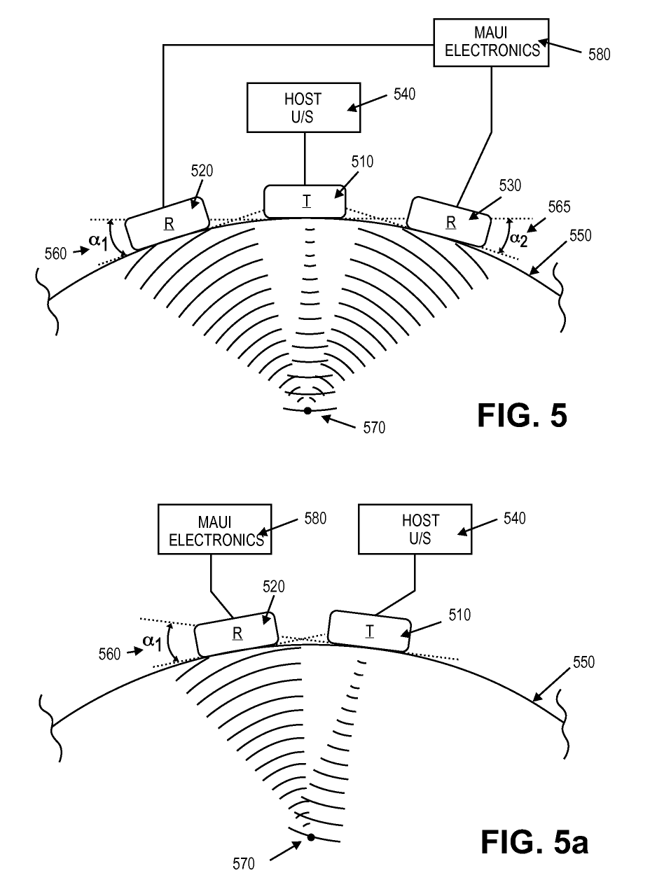

In FIG. 5, the entire probe, when assembled together, is used as an add-on device. It is connected to both an add-on instrument or MAUI Electronics 580 and to any host ultrasound system 540. The center array 510 can be used for transmit only. The outrigger arrays 520 and 530 can be used for receive only and are illustrated here on top of the skin line 550. Reflected energy off of scatterer 570 can therefore only be received by the outrigger arrays 520 and 530. The angulation of the outboard arrays 520 and 530 are illustrated as angles α₁ 560 or α₂ 565. These angles can be varied to achieve optimum beamforming for different depths or fields of view. α₁ and α₂ are often the same for outboard arrays, however, there is no requirement to do so. The MAUI Electronics can analyze the angles and accommodate unsymmetrical configurations. FIG. 5a demonstrates the right transducer 510 being used to transmit, and the other transducer 520 is being used to receive.

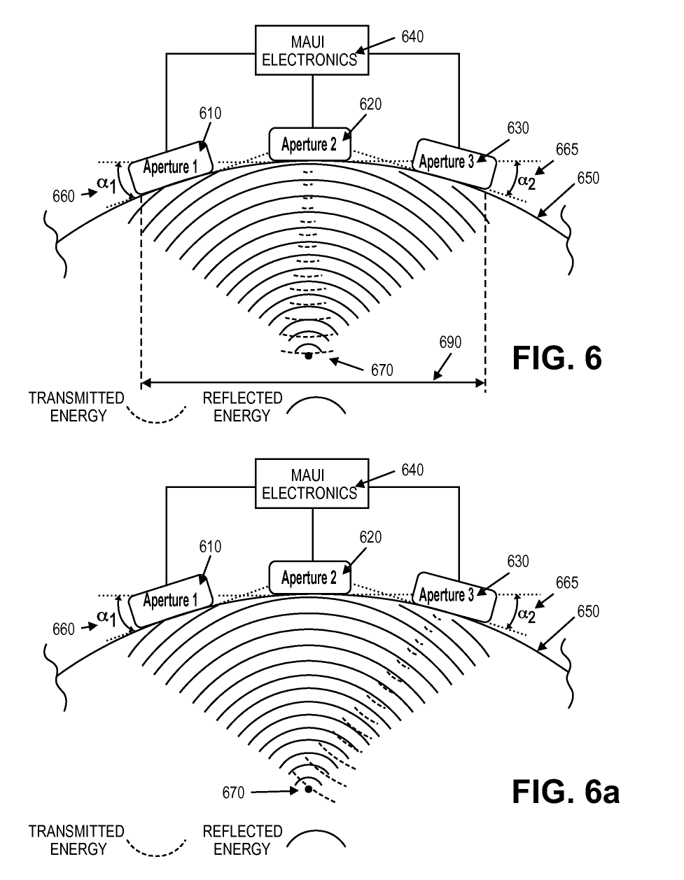

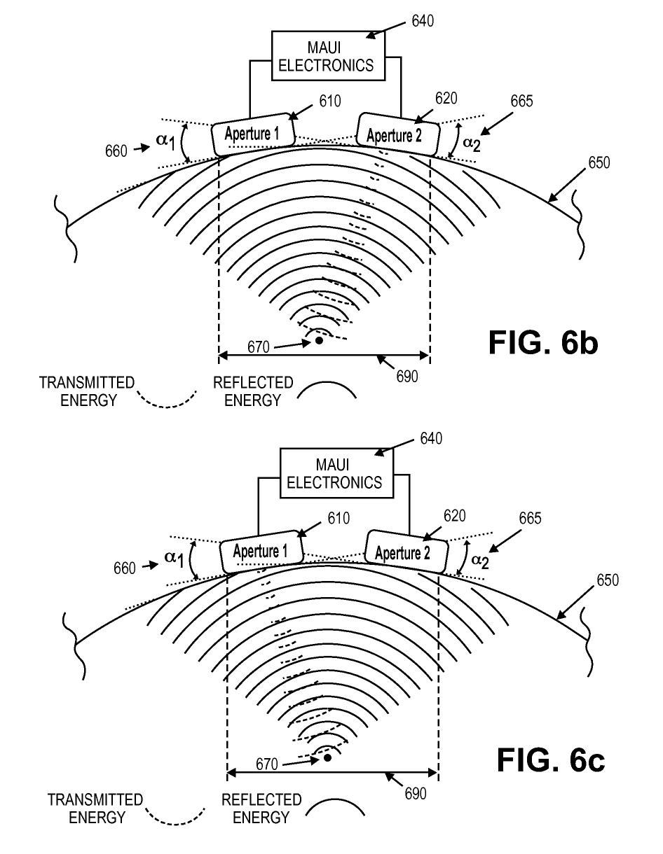

FIG. 6 is much like FIG. 5, except the Multiple Aperture Ultrasound Imaging System (MAUI Electronics) 640 used with the probe is a stand-alone system with its own on-board transmitter (i.e., no host ultrasound system is used). This system may use any element on any transducer 610, 620, or 630 for transmit or receive. The angulation of the outboard arrays 610 and 630 is illustrated as angle Δ 660. This angle can be varied to achieve optimum beamforming for different depths or fields of view. The angle is often the same for outboard arrays; however, there is no requirement to do so. The MAUI Electronics will analyze the angle and accommodate unsymmetrical configurations.

In this illustration, transmitted energy is coming from an element or small group of elements in Aperture 2 620 and reflected off of scatterer 670 to all other elements in all the apertures. Therefore, the total width 690 of the received energy is extends from the outermost element of Aperture 1 610 to the outmost element of Aperture 2 630. FIG. 6a shows the right array 610 transmitting, and all three arrays 610, 620 and 630 receiving. FIG. 6b shows elements on the left array 610 transmitting, and elements on the right array 620 receiving. Using one transducer for transmit only has advantages with regard to a lack of distortion due to variation in fat layer. In a standalone system, transmit and/or receive elements can be mixed in both or all three apertures.

FIG. 6b is much like FIG. 5a, except the Multiple Aperture Ultrasound Imaging System (MAUI Electronics) 640 used with the probe is a stand-alone system with its own on-board transmitter. This system may use any element on any array 610 or 620 for transmit or receive as is shown in FIG. 6c. As shown in either FIG. 6b or FIG. 6c, a transmitting array provides angle off from the target that adds to the collective aperture width 690 the same way two receive only transducers would contribute.

### General Assembly of a Multiple Aperture Transducer

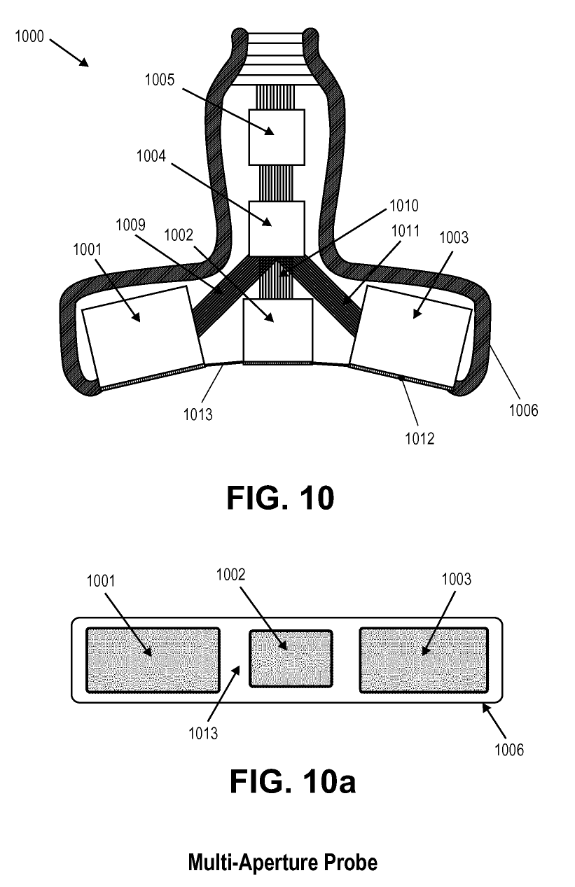

A multiple aperture ultrasound transducer has some distinguishing features. Elements or arrays can be physically separated and maintain different look angles toward the region of interest. Referring to FIG. 10, elements or arrays can each maintain a separate backing block 1001, 1002, and 1003, that keep the elements of a single aperture together, even though these arrays may ultimately share a common backing plate or probe shell 1006. There is no limit to the number of elements or arrays that can be used.

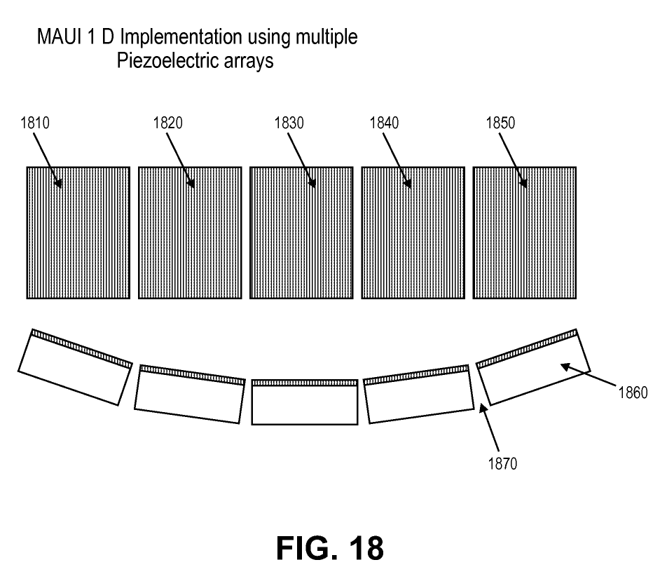

FIG. 18 shows a configuration of five arrays 1810, 1820, 1830, 1840, and 1850 that could be used in many of the probes illustrated. Also, there is no specific distance 1870 that must separate elements or arrays. Practitioners may falsely believe it is beneficial to construct a symmetrical probe; however, there is no requirement to do so. The MAUI electronics simply require the x, y, and z position of each element from a common origin, the origin can be located anywhere inside, above or below the probe. Once selected, the position of all elements are computed from the point of origin and loaded into the MAUI electronics.

Referring back to FIG. 1, the origin is centered in the middle of transmitting in probe 110, and the intersection of the x axis 150, y axis 160 and z axis 170 is illustrated. The freedom to construct probes using elements or arrays in oblong or off-center formats allows multiple aperture ultrasound transducers the ability to transmit and receive around undesired physiology which may degrade ultrasonic imaging (such as bone).

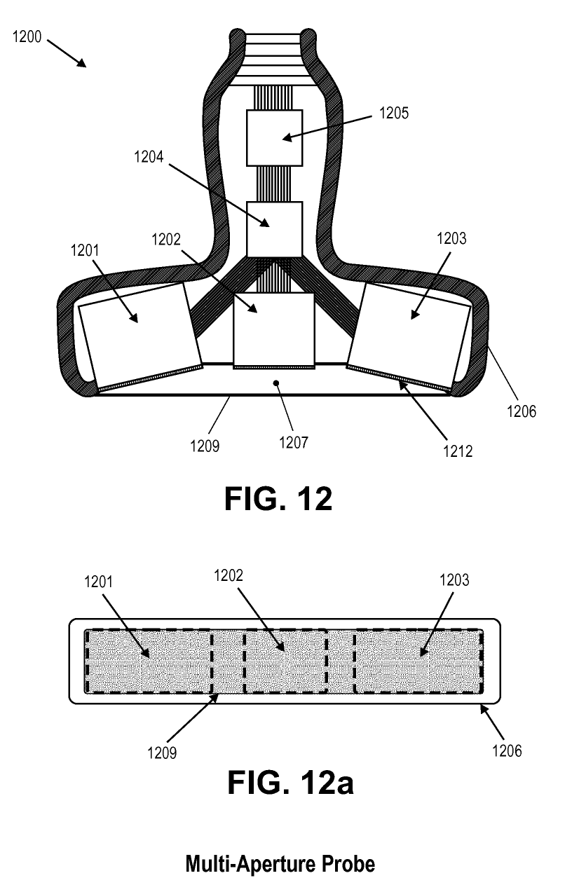

Another distinguishing feature is that elements on a backing block will maintain a common lens and flex connector. In FIG. 10, the right array 1003 has its own lens 1012 and flex connector 1011. The other arrays 1001 and 1002 each have their own lenses and flex connectors. A flex connector serves as a conduit for connectors from the array's backing block to what ultimately will become the cable connector to the host machine and, or MAUI electronics. The lens material used on a single aperture array 1212 in FIG. 12 may be independent of a common lens 1209 used for a collection of arrays contained in an enclosed space 1207.

Flex connection will need to be established to each backing block as is another distinguishing feature of multiple aperture ultrasound transducers. FIG. 10 illustrates three separate flex connectors 1009, 1010, 1011 coming off of independent arrays. The flex connectors are generally terminated and connected to microcoaxial cables before exiting the probe handle.

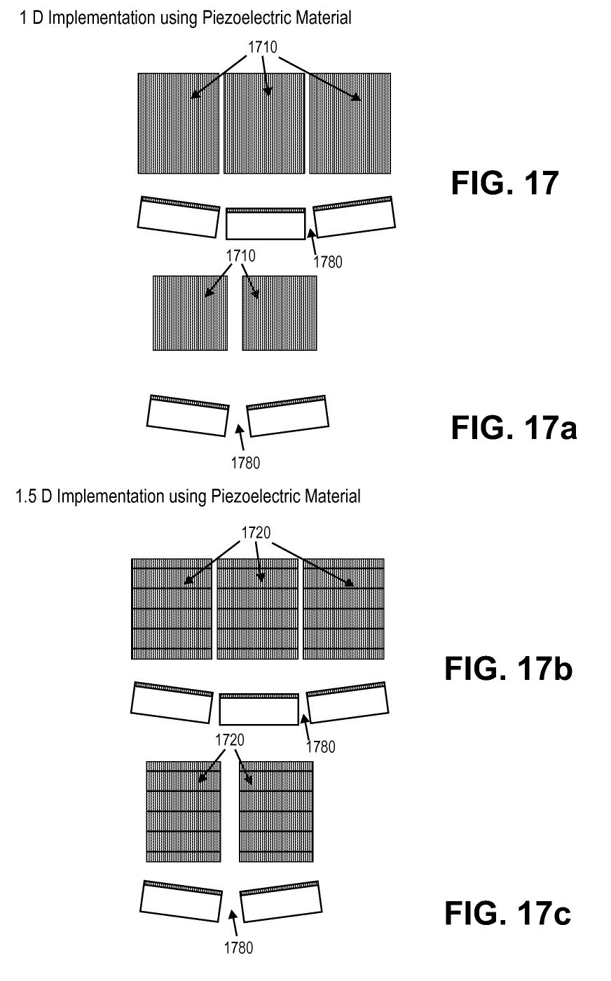

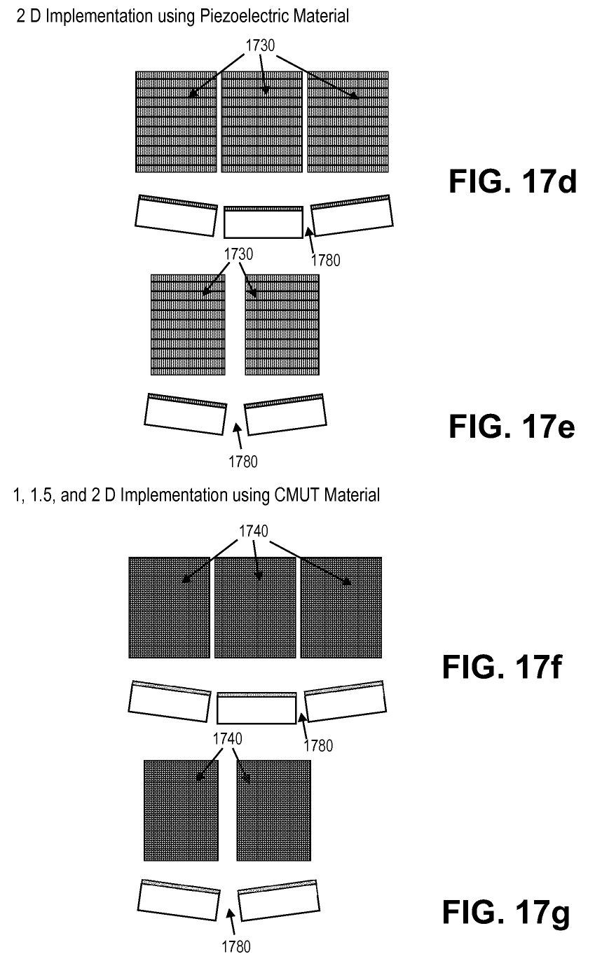

The construction of the transducers used in the probe apparatus is not a limitation of use in multi-aperture systems. FIG. 17 and FIG. 17a illustrate One Dimensional (1D) arrays 1710 spaced a distance 1780 apart that could be utilized in most MAUI Probe configurations, FIG. 17b and FIG. 17c illustrate One and Half Dimensional arrays 1720 spaced a distance 1780 apart can also be utilized in most MAUI Probe configurations, FIGS. 17d and 17e illustrate Two Dimensional (2D) arrays 1730 spaced a distance 1780 apart that could be used in all MAUI Probe configurations, as can CMUT transducers 1740 spaced a distance 1780 apart in FIG. 17f and FIG. 17g.

Examples of multi-aperture probe are shown below. These examples represent fabrication permutation of the multi-aperture probe.

### Multiple Aperture Cardiac Probe

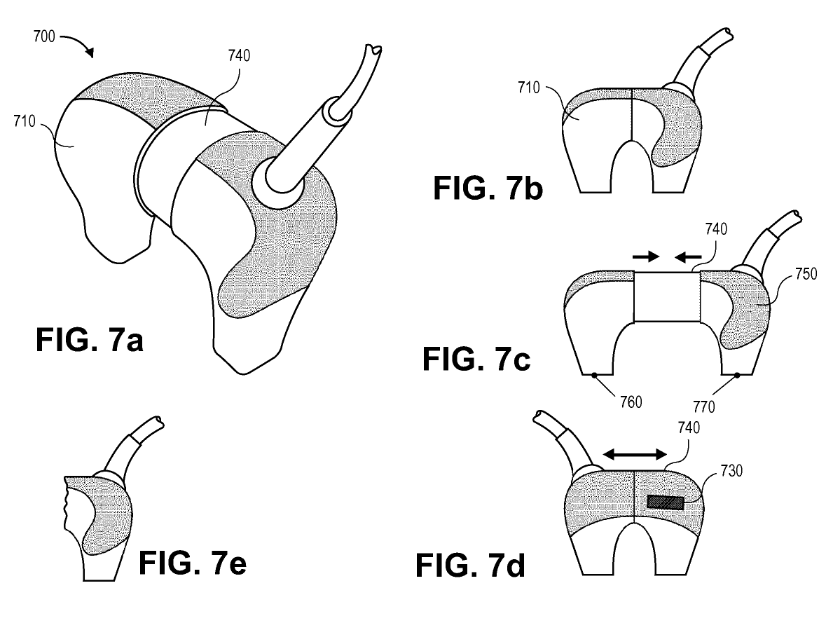

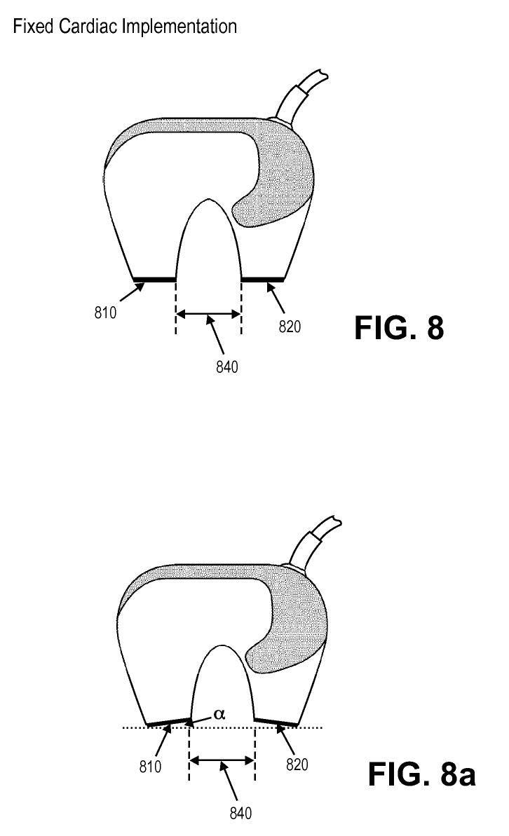

FIGS. 7 and 8 illustrate a multi-aperture probe 700 having a design and features that make it particularly well suited for cardiac applications. Referring to FIG. 7, the multi-aperture probe 700 can perform various movements to change the distance between adjacent arrays. One leg 710 of the probe encases elements or an array of elements 760, while the other leg 750 encases a separate group or array of elements 770. Referring to FIG. 7a, the probe can include an adjustment mechanism 740 configured to adjust the distance between the adjacent ultrasound transducer arrays. In some embodiments, a sensor inside the probe (not shown) can transmit mechanical position information of each of the arrays 760 and 770 back to the MAUI electronics.

The embodiment in FIG. 7d illustrates a thumb wheel 730 that is used to physically widen the probe. However, the technology is not restricted to mechanical adjustment of the probe. Wide arrays could be substituted, so that subsections of arrays 760 and 770 could electronically adjust the width of the probe.

FIG. 8 is a fixed position variant of the multi-aperture probe shown in FIG. 7-7e, having arrays 810 and 820. The width of the aperture 840 is fixed to accommodate different medical imaging applications. FIG. 8a demonstrates that transducers can be angled at an angle α for better beamforming characteristics just like any other MAUI probe.

### Arced Multiple Aperture Probe.

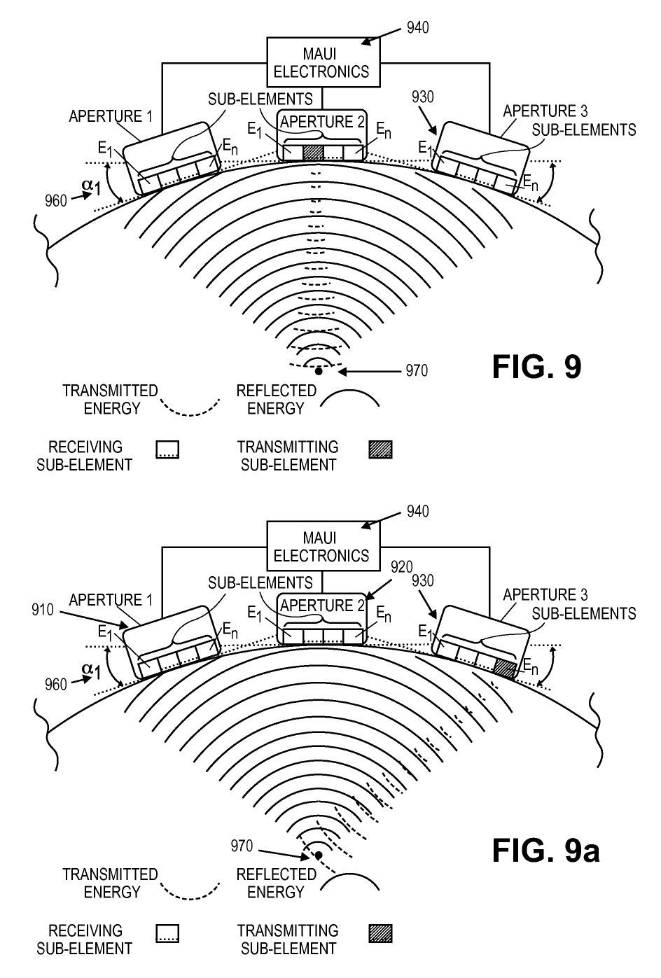

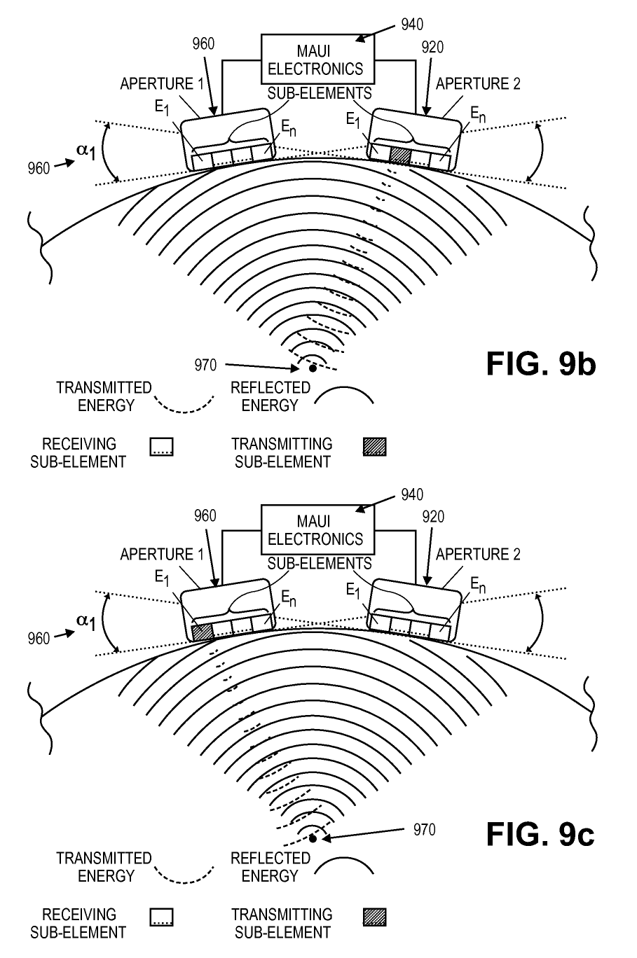

FIG. 10 is a diagram showing a multi-aperture probe 1000 with center array 1002 recessed to a point in line with the inboard edges of the outboard arrays 1001 and 1003. The lenses of the arrays are physically separated by a portion of the probe shell 1013. The outboard arrays can be canted at angles that are appropriate for ideal beamforming for different medical imaging applications. The probe 1000 can be attached to a controller (such as MAUI Electronics 940 in FIG. 9). FIG. 10 includes a transmit synchronizer module 1004 and probe position displacement sensor 1005. The transmit synchronization module 1004 is necessary to identify the start of pulse when the probe is used as an add-on device with a host machine transmitting. The probe displacement sensor 1005 can be an accelerometer or gyroscope that senses the three dimensional movement of the probe. The probe position displacement sensor can be configured to report the rate of angular rotation and lateral movement to the controller.

FIG. 10 includes outboard array 1001, the left most outboard array, and center array 1002, and outboard array 1003, the right most outboard array. In this embodiment, center array 1002 is positioned on a line that places the face of the array in line with the trailing edge of corners of outboard arrays 1001 and 1003, which can be installed at any desired inboard angle. This angle is established to optimize reception on echo information based on depth and area of interest.

In this embodiment, each of the arrays has its own lens 1012 that forms a seal with the outer shell of the probe housing 1006. The front surfaces of the lenses of arrays 1001, 1002, and 1003 combine with the shell support housing 1013 to form a concave arc. In some embodiments, transmit synchronization module 1004 is positioned directly above center array 1002, and configured to acquire reference transmit timing data. Probe position displacement sensor 1005 is positioned above the transmit synchronization module 1004. The displacement sensor transmits probe position and movement to the MAUI electronics for use in constructing 3D, 4D and volumetric images. Transducer shell 1006 encapsulates these arrays, modules and lens media.

FIG. 10a shows a frontal view of the separate lenses for arrays 1001, 1002, and 1003 within the probe shell 1006. The lenses are separated physically by a portion of the probe 1013.

### Straight Line Multiple Aperture Probe.

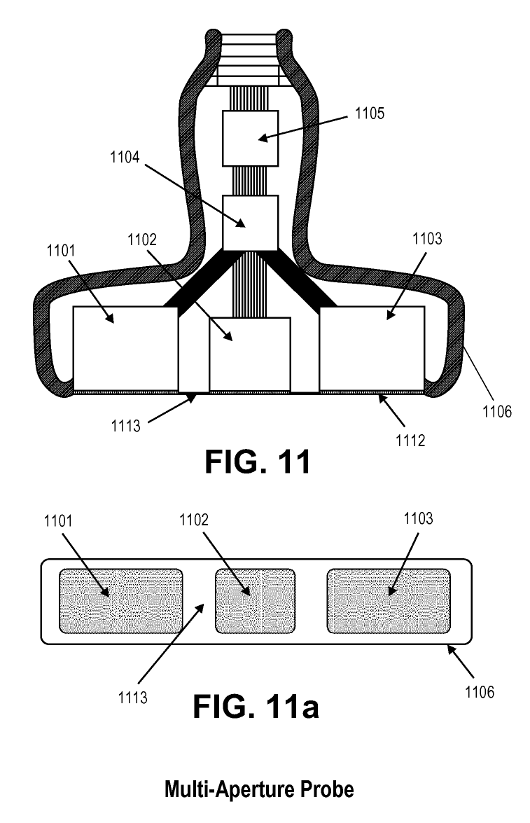

FIG. 11 is one embodiment of a multi-aperture probe 1100 with arrays configured in a horizontal plane and housed in shell 1106. FIG. 11 includes a transmit synchronizer module 1104 and probe position displacement sensor 1105. FIG. 11 shows array 1101, the left most outboard array, array 1102, the center array, and array 1103, the right most outboard array, positioned to form a straight edge surface. Also depicted in FIG. 11 is the probe's front wall 1113 separating the lenses 1112 of arrays 1101, 1102, and 1103. The transducer shell 2106 encapsulates these arrays, modules and the lens media.

FIG. 11a shows a view of the face or lens area. In FIG. 11a, the lenses of arrays 1101, 1102, 1103 are separated by the front wall 1113 of the probe shell.

The configuration shown in FIGS. 11 and 11a is one embodiment of a multi-aperture ultrasound probe 1100. It provides the advantage of having individual transducers come in direct contact with the patient over a wide area that cannot be easily covered with a convex array. Beamforming from linearly aligned arrays 1101, 1102 and 1103 may sometimes be more difficult.

### Offset Multiple Aperture Probe

FIG. 12 is a diagram showing a multi-aperture probe 1200 with center array 1202 recessed to a point in line with the trailing edges of the outboard arrays 1201 and 1203. However, the center array 1202 could be placed in any position within the enclosed area 1207. The probe can further include a unified lens and the outboard arrays can be canted at an angle within shell 1206. FIG. 12 includes a transmit synchronizer module 1204 and probe position displacement sensor 1205. The leading edge of arrays 1201 and 1203 are generally placed in contact with the surface of the transducer lens material 1209, which can cover the entire aperture of the transducer and provide a single lens opening for arrays 1201, 1202, and 1203.

Areas 207 contain suitable echo-lucent material to facilitate the transfer of ultrasound echo information with a minimum of degradation. Transducer shell 1206 can encapsulate these arrays, modules and the lens media.

FIG. 12a shows a view of the acoustic window. In FIG. 12a the acoustic window 1209 with outlines representing the mechanical position of array 1201 array 1202 and array 1203. The configuration shown in FIGS. 12 and 12a provides area of interest optimization for the Multi-Aperture Ultrasound Transducer for very high resolution near-field imaging in environments requiring enclosed or sterile standoffs while still gaining the advantage of multiple aperture imaging of the region of interest.

### Array Angles to Achieve Optimum Beamforming

In FIG. 9, the angle α₁960 is the angle between a line parallel to the elements of the left array 910 and an intersecting line parallel to the elements of the center array 920. Similarly, the angle α₂ 965 is the angle between a line parallel to the elements of the right array 930 and an intersecting line parallel to the elements of the center array 920. Angle α₁ and angle α₂ need not be equal; however, there are benefits in achieving optimum beamforming if they are nearly equal when angled inward toward the center elements or array 920. For the most part, the examples in FIGS. 10 through 12 illustrate a form of static or pre-set mechanical angulation.

In the illustrated examples, the angulation angle α can be approximately 12.5°. When α is at this angle, the effective aperture of the outboard sub arrays is maximized at a depth of about 10 cm from the tissue surface. The angulation angle α may vary within a range of values to optimize performance at different depths. At any depth, the effective aperture of the outrigger subarray is proportional to the sin of the angle between a line from this tissue scatterer to the center of the outrigger array and the surface of the array itself The angle α is chosen as the best compromise for tissues at a particular depth range.

The same solution taught in this disclosure is equally applicable for multi-aperture cardiac scanning, or for extended sparsely populated apertures for scans on other parts of the body.

### Omniplane Style Transesophogeal Implementation

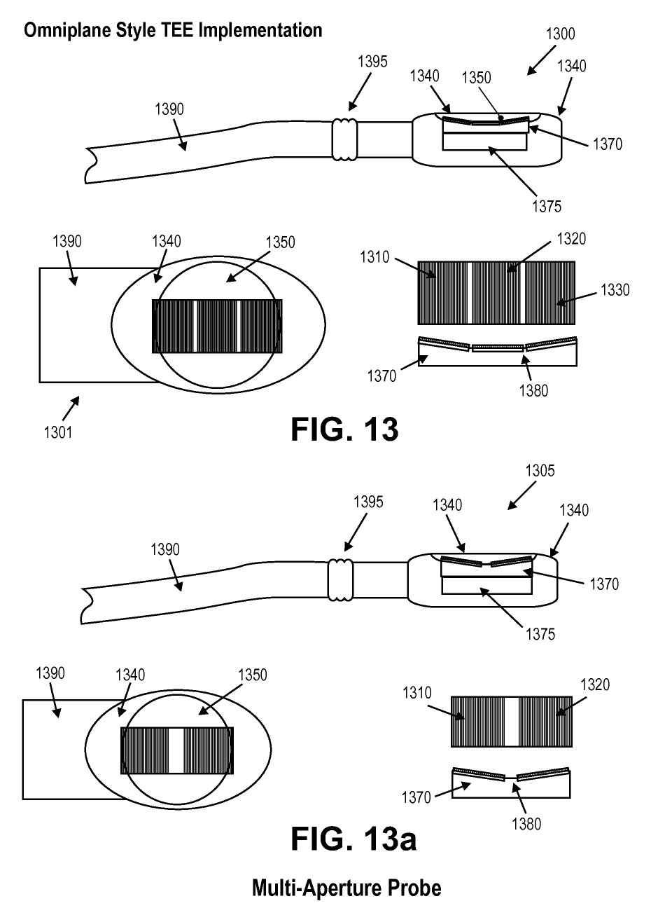

FIG. 13 is a diagram showing an Omniplane Style Transesophogeal probe sized and configured to be inserted into an esophagus of a patient, where 1300 is a side view and 1301 is a top view. In this embodiment, an enclosure 1350 contains multiple aperture arrays 1310, 1320 and 1330 that are located on a common backing plate 1370. The outer arrays 1310 and 1330 can be angled inwards at any angle, as described above. Even though positioned in a small space, the arrays are actually physically separated from each other a distance 1380, so that they can maintain separate apertures. The backing plate is mounted on a rotating turn table 1375 which can be operated mechanically or electrically to rotate the arrays. The enclosure 1350 contains suitable echo-lucent material to facilitate the transfer of ultrasound echo information with a minimum of degradation, and is contained by an acoustic window 1340. The operator may manipulate the probe through controls in the insertion tube 1390. The probe can move forward and aft and side to side beyond the bending rubber 1395.

FIG. 13a shows a view of Omniplane Style Transesophogeal probe using only two multiple aperture arrays. In this embodiment, an enclosure 1350 contains multiple aperture arrays 1310 and 1320 that are located on a common backing plate 1370. Both arrays 1310 and 1320 can be angled inwards, as described above. Even though positioned in a small space, the arrays are actually physically separated from each other a distance 1380, so that they can maintain separate apertures. The backing plate is mounted on a rotating turn table 1375 which can be operated mechanically or electrically to rotate the arrays. The enclosure 1350 contains suitable echo-lucent material to facilitate the transfer of ultrasound echo information with a minimum of degradation, and is contained by an acoustic window 1340. The operator may manipulate the probe through controls in the insertion tube 1390. The probe can move forward and aft and side to side beyond the bending rubber 1395.

The configuration shown in FIGS. 13 and 13a provides a Multi-Aperture Ultrasound Transducer for intracavity very high resolution imaging via the esophagus.

### Endo Rectal Probe Implementation

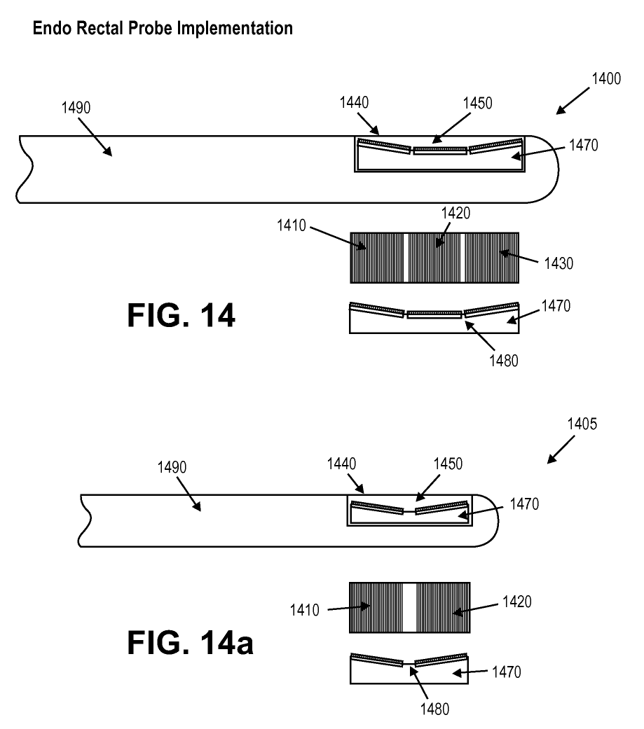

FIG. 14 is a diagram illustrating an Endo Rectal Probe 1400 sized and configured to be inserted into a rectum of a patient. In this embodiment, an enclosure 1450 contains multiple aperture arrays 1410, 1420 and 1430 that are located on a common backing plate 1470. The outer arrays 1410 and 1430 can be angled inwards at any angle, as described above. Even though positioned in a small space, the arrays are actually physically separated from each other a distance 1480, so that they can maintain separate apertures. The enclosure 1450 contains suitable echo-lucent material to facilitate the transfer of ultrasound echo information with a minimum of degradation, and is contained by an acoustic window 1440. The operator positions the probe manually. The probe shell 1490 houses the flex connectors and cabling in support of the multiple aperture arrays.

FIG. 14a shows a view an Endo Rectal Probe 1405 using only two arrays. In this embodiment, an enclosure 1450 contains multiple aperture arrays 1410 and 1420 that are located on a common backing plate 1470. Both arrays 1410 and 1420 can be angled inwards, as described above. Even though positioned in a small space, the arrays are actually physically separated from each other a distance 1480, so that they can maintain separate apertures. The enclosure 1450 contains suitable echo-lucent material to facilitate the transfer of ultrasound echo information with a minimum of degradation, and is contained by an acoustic window 1440. The operator positions the probe manually. The probe shell 1490 houses the flex connectors and cabling in support of the multiple aperture arrays.

The configuration shown in FIGS. 14 and 14a provides a Multi-Aperture Ultrasound Transducer for intracavity very high resolution imaging via the rectum or other natural lumens.

### Endo Vaginal Probe

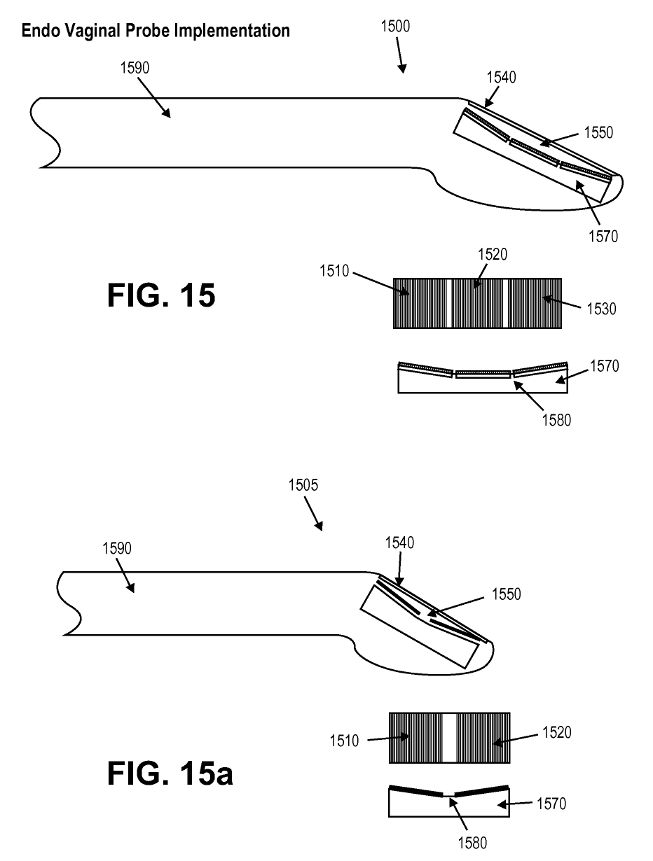

FIG. 15 is a diagram illustrating an Endo Vaginal Probe 1500 sized and configured to be inserted into a vagina of a patient. In this embodiment, an enclosure 1550 contains multiple aperture arrays 1510, 1520 and 1530 that are located on a common backing plate 1570. The outer arrays 1510 and 1530 can be angled inwards at any angle, as described above. Even though positioned in a small space, the arrays are actually physically separated from each other a distance 1580, so that they can maintain separate apertures. The enclosure 1550 contains suitable echo-lucent material to facilitate the transfer of ultrasound echo information with a minimum of degradation, and is contained by an acoustic window 1540. The operator positions the probe manually. The probe shell 1590 houses the flex connectors and cabling in support of the multiple aperture arrays.

FIG. 15a shows a view an Endo Vaginal Probe 1505 using only two arrays. In this embodiment, an enclosure 1550 contains multiple aperture arrays 1510 and 1520 that are located on a common backing plate 1570. Both arrays 1510 and 1520 can be angled inwards, as described above. Even though positioned in a small space, the arrays are actually physically separated from each other a distance 1580, so that they can maintain separate apertures. The enclosure 1550 contains suitable echo-lucent material to facilitate the transfer of ultrasound echo information with a minimum of degradation, and is contained by an acoustic window 1540. The operator positions the probe manually. The probe shell 1590 houses the flex connectors and cabling in support of the multiple aperture arrays.

The configuration shown in FIGS. 15 and 15a provides a Multi-Aperture Ultrasound Transducer for intracavity very high resolution imaging via the vagina.

### Intravenous Ultrasound Probe Implementation

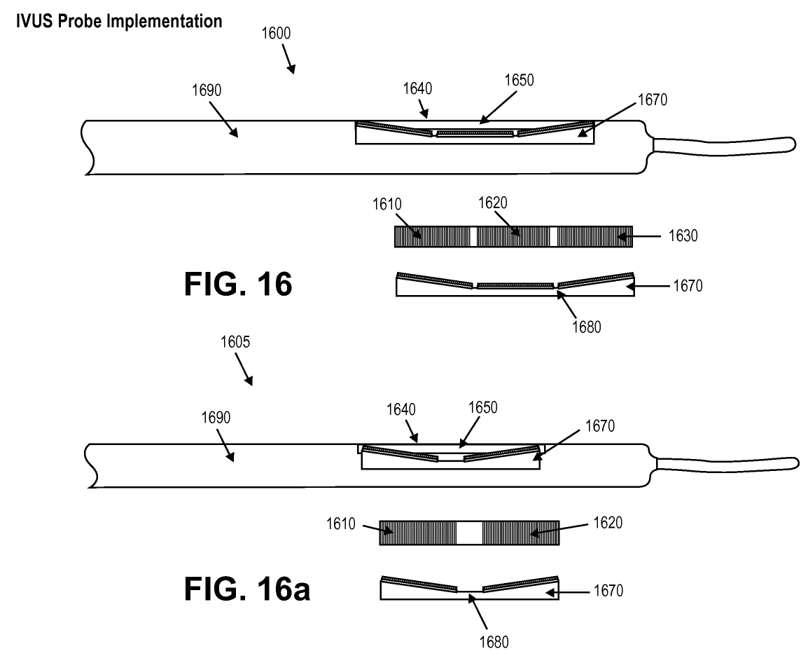

FIG. 16 is a diagram showing an Intravenous Ultrasound Probe (IVUS) probe sized and configured to be inserted into a vessel of a patient. In this embodiment, an enclosure 1650 contains multiple aperture arrays 1610, 1620 and 1630 that are located on a common backing plate 1670. The outer arrays 1610 and 1630 can be angled inwards at any angle, as described above. Even though positioned in a small space, the arrays are actually physically separated from each other a distance 1680, so that they can maintain separate apertures. The enclosure 1650 contains suitable echo-lucent material to facilitate the transfer of ultrasound echo information with a minimum of degradation, and is contained by an acoustic window 1640. The operator may manipulate the probe through controls attached to and inside of the catheter 1690. The probe is placed in a vessel and can be rotated in a circular motion as well as fore and aft.

FIG. 16a shows a view of Intravenous Ultrasound Probe (IVUS) probe using only two multiple aperture arrays. In this embodiment, an enclosure 1650 contains multiple aperture arrays 1610 and 1620 that are located on a common backing plate 1670. Both arrays 1610 and 1620 can be angled inwards at any angle, as described above. Even though positioned in a small space, the arrays are actually physically separated from each other a distance 1680, so that they can maintain separate apertures. The enclosure 1650 contains suitable echo-lucent material to facilitate the transfer of ultrasound echo information with a minimum of degradation, and is contained by an acoustic window 1640. The operator may manipulate the probe through controls attached to and inside of the catheter 1690. The probe is placed in a vessel and can be rotated in a circular motion as well as fore and aft.

The configuration shown in FIGS. 16 and 16a provides a Multi-Aperture Ultrasound Transducer for intravenous imaging via a blood filled vessel.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A multi-aperture ultrasound probe, comprising:
a probe shell;
a first ultrasound transducer array disposed in the shell and having a plurality of transducer elements, wherein at least one of the plurality of transducer elements of the first ultrasound transducer array is configured to transmit an ultrasonic pulse;
a second ultrasound transducer array disposed in the shell and being physically separated from the first ultrasound transducer array, the second ultrasound transducer array having a plurality of transducer elements, wherein at least one of the plurality of transducer elements of the second ultrasound transducer array is configured to receive an echo return of the ultrasonic pulse.

2. The multi-aperture ultrasound probe of claim 1 wherein the second ultrasound transducer array is angled towards the first ultrasound transducer array.

3. The multi-aperture ultrasound probe of claim 1 wherein the second ultrasound transducer array is angled in the same direction as the first ultrasound transducer array.

4. The multi-aperture ultrasound probe of claim 1 wherein at least one of the plurality of transducer elements of the first ultrasound transducer array is configured to receive an echo return of the ultrasonic pulse.

5. The multi-aperture ultrasound probe of claim 1 wherein at least one of the plurality of transducer elements of the second ultrasound transducer array is configured to transmit an ultrasonic pulse.

6. The multi-aperture ultrasound probe of claim 4 wherein at least one of the plurality of transducer elements of the second ultrasound transducer array is configured to transmit an ultrasonic pulse.

7. The multi-aperture ultrasound probe of claim 1 wherein the shell further comprises an adjustment mechanism configured to adjust the distance between the first and second ultrasound transducer arrays.

8. The multi-aperture ultrasound probe of claim 1 further comprising a third ultrasound transducer array disposed in the shell and being physically separated from the first and second ultrasound transducer arrays, the third ultrasound transducer array having a plurality of transducer elements, wherein at least one of the plurality of transducer elements of the third ultrasound transducer array is configured to receive an echo return of the ultrasonic pulse.

9. The multi-aperture ultrasound probe of claim 8 wherein the first ultrasound transducer array is positioned near the center of the shell and the second and third ultrasound transducer arrays are positioned on each side of the first ultrasound transducer array.

10. The multi-aperture ultrasound probe of claim 9 wherein the second and third ultrasound transducer arrays are angled towards the first ultrasound transducer array.

11. The multi-aperture ultrasound probe of claim 10 wherein the first ultrasound transducer array is recessed within the shell

12. The multi-aperture ultrasound probe of claim 11 wherein the first ultrasound transducer array is recessed within the shell to be approximately aligned with an inboard edge of the second and third ultrasound transducer arrays.

13. The multi-aperture ultrasound probe of claim 10 wherein the first, second, and third ultrasound transducer arrays each comprise a lens that forms a seal with the shell.

14. The multi-aperture ultrasound probe of claim 13 wherein the lenses form a concave arc.

15. The multi-aperture ultrasound probe of claim 11 further comprising a single lens opening for the first, second, and third ultrasound transducer arrays.

16. The multi-aperture ultrasound probe of claim 1 wherein the shell is sized and configured to be inserted into an esophagus of a patient.

17. The multi-aperture ultrasound probe of claim 1 wherein the shell is sized and configured to be inserted into a rectum of a patient.

18. The multi-aperture ultrasound probe of claim 1 wherein the shell is sized and configured to be inserted into a vagina of a patient.

19. The multi-aperture ultrasound probe of claim 1 wherein the shell is sized and configured to be inserted into a vessel of a patient.

20. The multi-aperture ultrasound probe of claim 1 wherein the plurality of transducer elements of the first ultrasound transducer can be grouped and phased to transmit a focused beam.

21. The multi-aperture ultrasound probe of claim 1 wherein at least one of the plurality of transducer elements of the first ultrasound transducer are configured to produce a semicircular pulse to insonify an entire slice of a medium.

22. The multi-aperture ultrasound probe of claim 1 wherein at least one of the plurality of transducer elements of the first ultrasound transducer are configured to produce a semispherical pulse to insonify an entire volume of the medium.

23. The multi-aperture ultrasound probe of claim 1 wherein the first and second transducer arrays include separate backing blocks.

24. The multi-aperture ultrasound probe of claim 23 wherein the first and second transducer arrays further comprise a flex connector attached to the separate backing blocks.

25. The multi-aperture ultrasound probe of claim 1 further comprising a probe position displacement sensor configured to report a rate of angular rotation and lateral movement to a controller.

26. The multi-aperture ultrasound probe of claim 1 wherein the first ultrasound transducer array comprises a host ultrasound probe, the multi-aperture ultrasound probe further comprising a transmit synchronizer device configured to report a start of transmit from the host ultrasound probe to a controller.
